(19) United States
(12) Patent Application Publication
(10) Pub. No.: US 2014/0100597 A1
(43) Pub. Date: Apr. 10, 2014

(54) SPECIAL OPENING AORTIC EMBOLIC PROTECTION DEVICE FOR STRUCTURAL HEART PROCEDURES

(71) Applicant: BOSTON SCIENTIFIC SCIMED, INC., Maple Grove, MN (US)

(72) Inventors: Huisun Wang, Maple Grove, MN (US); James M. Anderson, Fridley, MN (US)

(21) Appl. No.: 14/049,385

(22) Filed: Oct. 9, 2013

Related U.S. Application Data
(60) Provisional application No. 61/711,344, filed on Oct. 9, 2012.

(51) Int. Cl. A61F 2/01

(52) U.S. Cl. CPC A61F 2/01
USPC 606/200

(57) ABSTRACT

A medical device may include an embolic protection device including a distal filter element, a proximal filter element, and a deflector element disposed between and spacing apart the distal filter element and the proximal filter element. An embolic protection device may include a longitudinally-oriented mouth extending from the distal filter element to the proximal filter element. A method of providing embolic protection in an aortic arch may include inserting a guidewire from a left subclavian artery through the aortic arch into a brachiocephalic artery, deploying a distal filter element within the brachiocephalic artery such that a mouth overlies an ostium of the brachiocephalic artery, deploying a deflector element over an ostium of a carotid artery disposed between the brachiocephalic artery and the left subclavian artery, and deploying a proximal filter element within the left subclavian artery such that a mouth overlies an ostium of the left subclavian artery.

### CROSS-REFERENCE TO RELATED APPLICATIONS

This application claims priority to U.S. Provisional Application No. 61/711,344 filed Oct. 9, 2012.

### TECHNICAL FIELD

The disclosure relates generally to percutaneous medical devices and more particularly to embolic protection devices for use in the aortic arch to protect carotid arteries.

### BACKGROUND

Preventing emboli and other debris from entering the carotid arteries (i.e., the right common carotid artery and/or the brachiocephalic artery, and the left common carotid artery) by way of the aorta reduces the incidence of ischemic stroke. Emboli and other debris in the aorta come from several sources. These sources include: 1) aortic atheroma which detaches from the wall of the aorta due to various reasons including incising, clamping, and/or clamp release of the aorta during surgery; 2) debris released during surgery on the heart such as the installation of a replacement heart valve or to access the left atrial appendage; 3) thrombus which forms in the right atrium resulting from atrial fibrillation; 4) thrombus which forms on ventricular assist devices; 5) venous thrombus which passes into the left ventricle through a patent foramen ovale or other arteriovenous shunt; and 6) other less common sources.

A variety of intravascular filtering means are known in the art and may consist of a flexible metallic grid, a flexible synthetic or plastic grid, a weave of synthetic filaments, or a non-degradable or possibly biodegradable textile cloth, often supported by a basket or funnel shaped frame which may be deployed within the lumen of a vessel to be protected.

There are fewer intravascular devices designed for arterial and especially aortic filtration. A filter that functions in arteries must address additional concerns because of the hemodynamic differences between arteries and veins. Arteries are much more flexible and elastic than veins and, in the arteries, blood flow is pulsatile with large pressure variations between systolic and diastolic flow. These pressure variations cause the artery walls to expand and contract. Thus, filtering devices must be able to expand and contract along with the lumen of the aorta to which they may be anchored. In addition, intravascular devices for aortic filtration and/or diversion of emboli typically occlude a significant portion of the lumen of the aorta rendering them unsatisfactory for use in combination with other intravascular interventional procedures.

The problem of preventing emboli from reaching the cerebral vasculature has thus far not been adequately addressed. Therefore, a need exists for new devices and methods to prevent embolic material from entering the carotid/cerebral arteries, while maintaining peripheral blood flow from the heart to the descending aorta.

### SUMMARY

A medical device for aortic embolic protection may include a guidewire, an embolic protection device slideably disposed about the guidewire, the embolic protection device including a distal filter element having a mouth, a proximal filter element having a mouth, and a deflector element disposed between and spacing apart the mouth of the distal filter element and the mouth of the proximal filter element, and a catheter configured to receive the embolic protection device therein, the catheter being slideably disposed over the guidewire.

A medical device for aortic embolic protection may include a guidewire, an embolic protection device slideably disposed about the guidewire, the embolic protection device including a distal filter element, a proximal filter element, and a deflector element disposed between and spacing apart the distal filter element and the proximal filter element, wherein the embolic protection device includes a longitudinally-oriented mouth extending from the distal filter element to the proximal filter element, and a catheter configured to receive the embolic protection device therein, the catheter being slideably disposed over the guidewire.

A method of providing embolic protection in an aortic arch may include inserting a guidewire from a left subclavian artery through the aortic arch into a brachiocephalic artery, advancing a catheter having an embolic protection device disposed therein over the guidewire from the left subclavian artery through the aortic arch into the brachiocephalic artery, the embolic protection device including a distal filter element having a mouth, a proximal filter element having a mouth, and a deflector element disposed between and spacing apart the mouth of the distal filter element and the mouth of the proximal filter element, withdrawing the catheter proximally and deploying the distal filter element within the brachiocephalic artery such that the mouth of the distal filter element overlies an ostium of the brachiocephalic artery, after deploying the distal filter element, further withdrawing the catheter proximally and deploying the deflector element over an ostium of a carotid artery disposed between the brachiocephalic artery and the left subclavian artery, and after deploying the deflector element, further withdrawing the catheter proximally and deploying the proximal filter element within the left subclavian artery such that the mouth of the proximal filter element overlies an ostium of the left subclavian artery.

### BRIEF DESCRIPTION OF THE DRAWINGS

FIG. 1 is a schematic partial cross-sectional view of an aortic arch;

FIG. 2 is a partial schematic view of an example medical device;

FIGS. 3-8 illustrate deployment of an example medical device in an aortic arch;

FIG. 9 is a partial schematic view of an example medical device deployed in an aortic arch;

FIG. 10 is a partial schematic view of the example medical device of FIG. 9 in preparation for recapture; and

FIG. 11 is a partial schematic view of an example medical device deployed in an aortic arch.

While the invention is amenable to various modifications and alternative forms, specifics thereof have been shown by way of example in the drawings and will be described in greater detail below. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following description should be read with reference to the drawings wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

The terms “upstream” and “downstream” refer to a position or location relative to the direction of blood flow through a particular element or location, such as a vessel (i.e., the aorta) or vessel lumen, a heart valve (i.e., the aortic valve), and the like.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range, including the endpoints (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

Although portions of this disclosure may refer to covering one or the other of the ostium of the brachiocephalic artery and the ostium of the right common carotid artery, it will be understood that those individual terms as used herein are to encompass variations in the anatomy which may be found in that region. Thus a device which covers the ostium of the brachiocephalic artery is to be viewed as covering an individual anatomy in which the right common carotid artery branches from the brachiocephalic artery after it leaves the aorta as well as an individual anatomy in which the right common carotid artery connects directly to the aorta. In either anatomy, the disclosed embolic protection device should be viewed as protecting at least the right common carotid artery and the left common carotid artery.

As intravascular approaches to heart surgery, such as aortic valve replacement, have become more common, it has become more desirable to provide devices which filter or divert emboli or other debris from reaching the carotid arteries, and thereby to reduce the incidence of ischemic stroke, while at the same time maintaining access to the heart tissue for other medical devices.

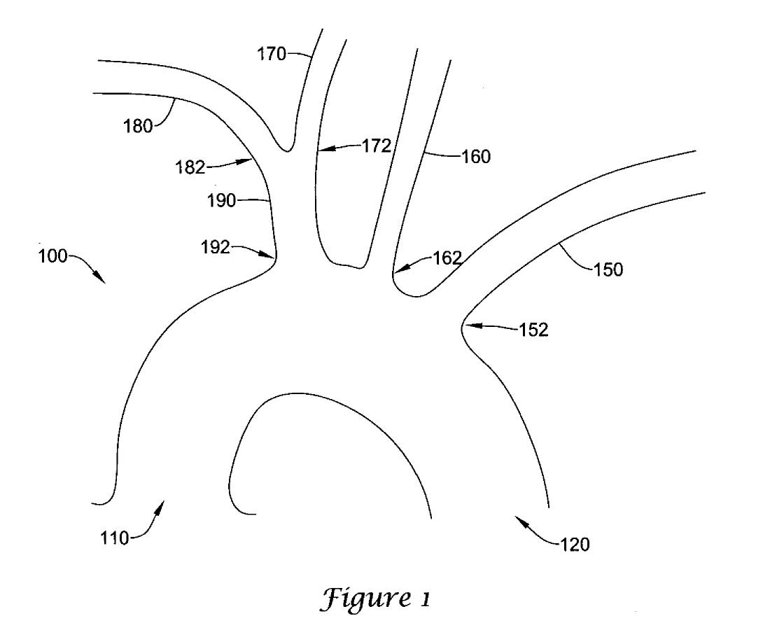

FIG. 1 schematically illustrates an aortic arch 100. An aortic arch 100 may include an aortic valve at a first end 110 disposed between the aortic arch 100 and a heart (not shown), an outgoing portion leading to a patient's aorta for distributing oxygenated blood throughout the body at a second end 120, and several arteries branching off of the “top” of the aortic arch 100. The arteries branching off of the “top” of the aortic arch 100 may include a left subclavian artery 150, a left common carotid artery 160, and a brachiocephalic artery 190, which may include a right common carotid artery 170 and a right subclavian artery 180 branching therefrom. In some patients and/or embodiments, the right common carotid artery 170 branches off of the brachiocephalic artery 190 after the brachiocephalic artery 190 branches from the aortic arch 100. In some patients and/or embodiments, the right common carotid artery 170 may branch directly from the aortic arch 100.

Each artery branching from the aortic arch 100 includes an ostium or opening where the artery leaves or branches from the aortic arch 100. As such, the left subclavian artery 150 includes an ostium 152 at a mouth of the left subclavian artery 150 into or from the aortic arch 100. The left common carotid artery 160 includes an ostium 162 at a mouth of the left common carotid artery 160 into or from the aortic arch 100. The right subclavian artery 180 includes an ostium 182 at a mouth of the right subclavian artery 180 into or from brachiocephalic artery 190. The right common carotid artery 170 includes an ostium 172 at a mouth of the right common carotid artery 170 into or from, for the purpose of illustration, the brachiocephalic artery 190. As understood from the discussion above, while not explicitly illustrated, the right common carotid artery 170 may include an ostium 172 at a mouth of the right common carotid artery 170 directly into or from the aortic arch 100. The brachiocephalic artery 190 includes an ostium 192 at a mouth of the brachiocephalic artery 190 into or from the aortic arch 100.

Generally, the left common carotid artery 160 and the right common carotid artery 170 are disposed between the left subclavian artery 150 and the right subclavian artery 180, as may be seen in FIG. 1. As is known in the art, proper blood flow moves from the aortic valve at the first end 110 away from the heart (not shown) and toward the branching arteries and aorta at the second end 120. Accordingly, blood normally flows from the aortic arch 100 through the ostium and into each artery for carriage to the brain and/or other organs or portions of the body.

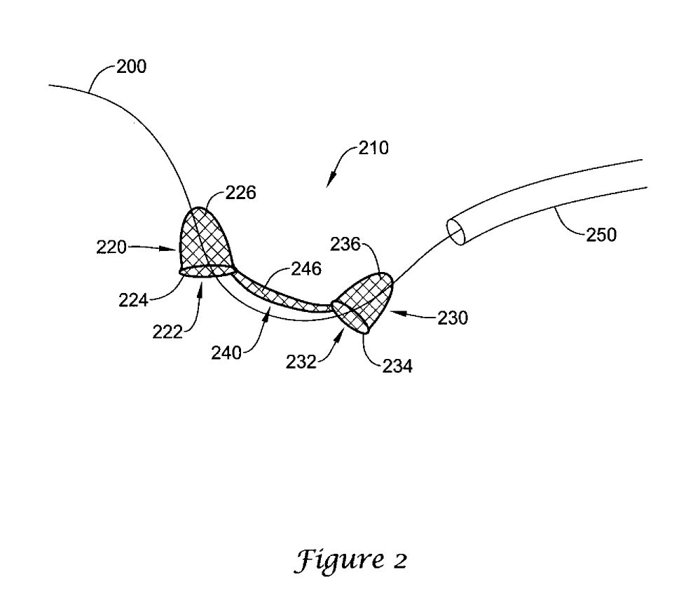

As illustrated in FIG. 2, a medical device for aortic embolic protection may include a guidewire 200, an embolic protection device 210, and a catheter 250. In some embodiments, the embolic protection device 210 may be slideably disposed about, over, or along the guidewire 200. In some embodiments, the catheter 250 may be slideably disposed over the guidewire 200. In some embodiments, the embolic protection device 210 may be disposed within a lumen of the catheter 250 prior to percutaneous insertion, and may be deployed from the catheter 250 at a treatment site, where the treatment site may be within or adjacent to the aortic arch 100. In some embodiments, the catheter 250 may be configured to receive the embolic protection device 210 therein in a substantially linear fashion, as will become apparent from the discussion below.

In some embodiments, the embolic protection device 210 may include a distal filter element 220 having a mouth 222, a proximal filter element 230 having a mouth 232, and a deflector element 240 fixedly attached to and disposed between the distal filter element 220 and the proximal filter element 230. In some embodiments, the deflector element 240 may space apart the mouth 222 of the distal filter element 220 and the mouth 232 of the proximal filter element 230.

In some embodiments, the distal filter element 220 is adapted to be placed in the ostium 192 of the brachiocephalic artery 190, and the mouth 222 of the distal filter element 220 is sized and adapted to cover and/or overlie the ostium 192 of the brachiocephalic artery 190 such that embolic material in the aortic arch 100 is permitted to enter the distal filter element 220 through the mouth 222 of the distal filter element 220. The distal filter element 220 may include a self-expanding support hoop 224 forming the mouth 222 and a blood permeable filter material 226 attached thereto adapted to capture the embolic material therein.

In some embodiments, the proximal filter element 230 is adapted to be placed in the ostium 152 of the left subclavian artery 150, and the mouth 232 of the proximal filter element 230 is sized and adapted to cover and/or overlie the ostium 152 of the left subclavian artery 150 such that embolic material in the aortic arch 100 is permitted to enter the proximal filter element 230 through the mouth 232 of the proximal filter element 230. The proximal filter element 230 may include a self-expanding support hoop 234 forming the mouth 232 and a blood permeable filter material 236 attached thereto adapted to capture the embolic material therein.

In some embodiments, the deflector element 240 is sized and adapted to be placed over an ostium of a carotid artery disposed between the brachiocephalic artery 190 and the left subclavian artery 150, for example, the ostium 162 of the left common carotid artery 160 and/or the ostium 172 of the right common carotid artery 170 in anatomies where the right common carotid artery 170 connects directly to the aortic arch 100. The deflector element 240 may be sized to substantially cover and/or overlie the ostium 162 of the left common carotid artery 160 and/or the ostium 172 of the right common carotid artery 170. The deflector element 240 may include or be formed from a blood permeable filter material 246.

The blood permeable filter material(s) 226, 236, 246 may include or be formed from a porous membrane, a woven fabric or mesh, or other suitable filtering structure(s). The blood permeable material(s) 226, 236, 246 may include holes or apertures therethrough sized and/or shaped to allow to blood to pass through the blood permeable material largely unimpeded while preventing harmful emboli from passing through the blood permeable material.

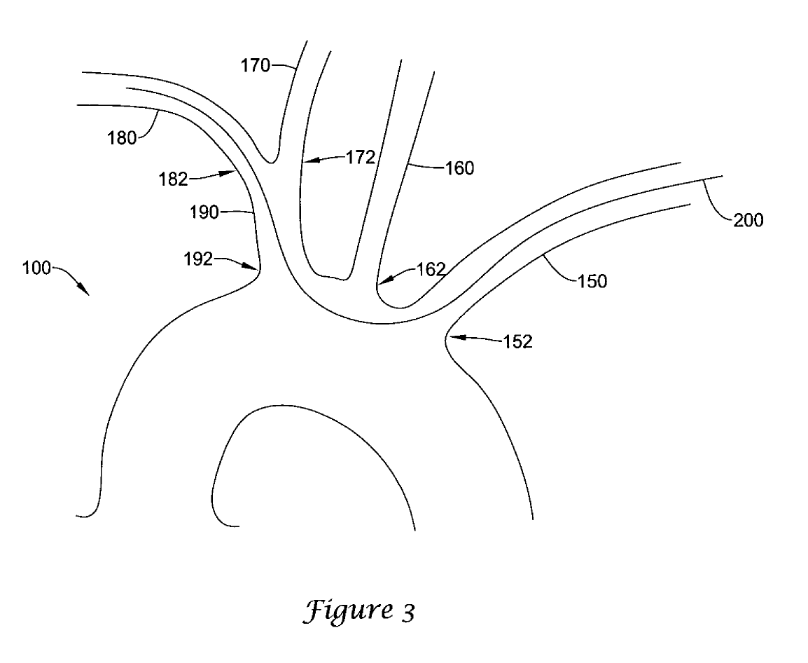

FIG. 3 illustrates the guidewire 200 extending from the left subclavian artery 150 through the aortic arch 100 and into the right subclavian artery 180 via the brachiocephalic artery 190. For the purpose of illustration, use of the medical device(s) described herein will be explained as though the medical device is inserted via the left subclavian artery 150. However, one of skill in the art will appreciate that the medical device may be inserted via the right subclavian artery 180, via the aorta, or other suitable routes.

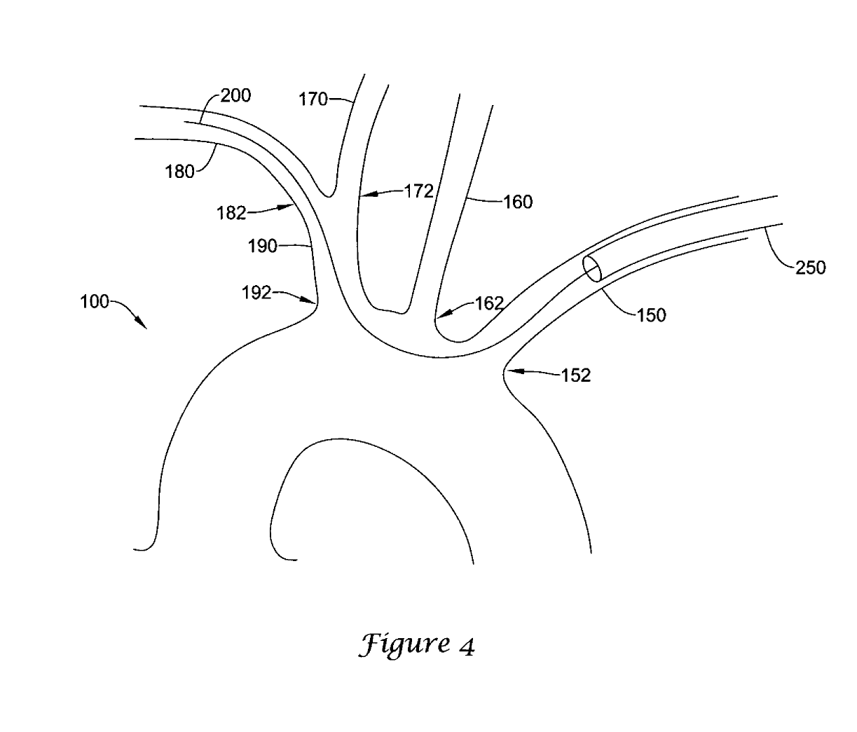

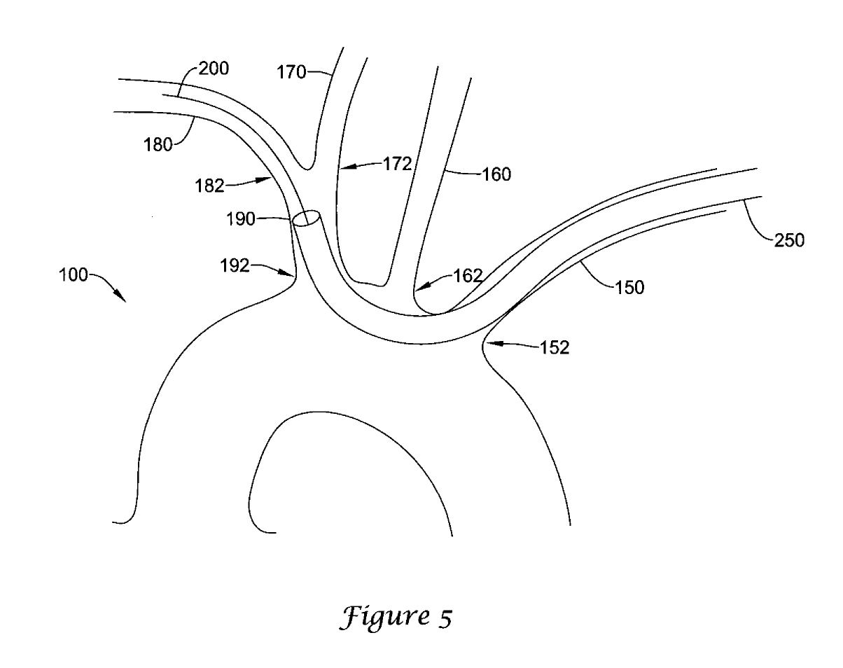

FIG. 4 illustrates the catheter 250 being advanced along or over the guidewire 200 through the left subclavian artery 150 and approaching the aortic arch 100. The catheter 250 may include an embolic protection device 210 disposed within a lumen of the catheter 250. FIG. 5 illustrates the catheter 250 advanced through the aortic arch 100 and into the brachiocephalic artery 190, where the distal filter element 220 is to be deployed.

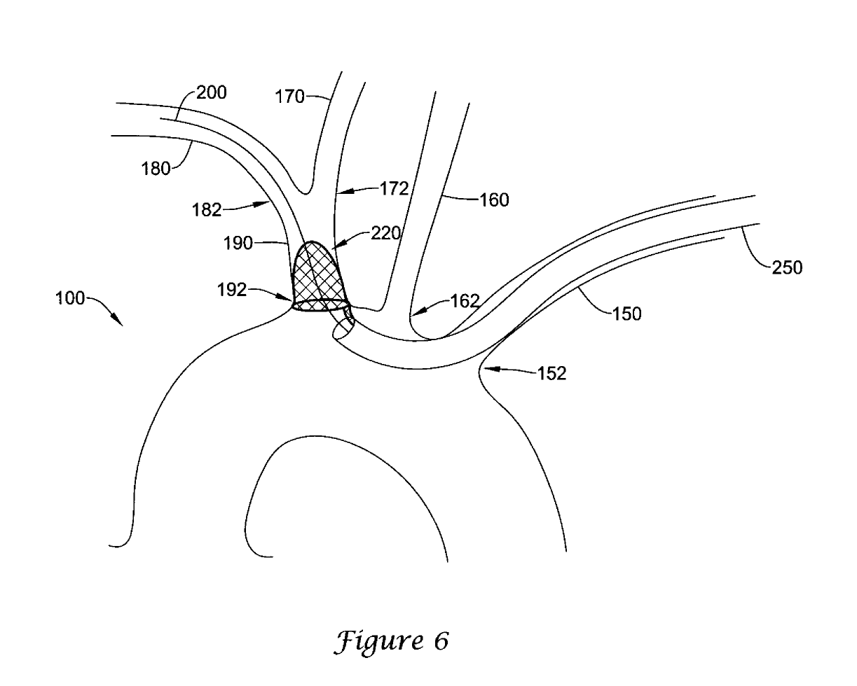

FIG. 6 illustrates withdrawing the catheter 250 proximally and deploying the distal filter element 220 within the brachiocephalic artery 190 such that the mouth 222 of the distal filter element 220 covers and/or overlies the ostium 192 of the brachiocephalic artery 190. In some embodiments, the embolic protection device 210 may be disposed within the catheter 250 in a substantially linear arrangement. Due to the substantially linear arrangement of the embolic protection device 210 within catheter 250, proximal refraction of the catheter 250 deploys the distal filter element 220 prior to deploying the deflector element 240.

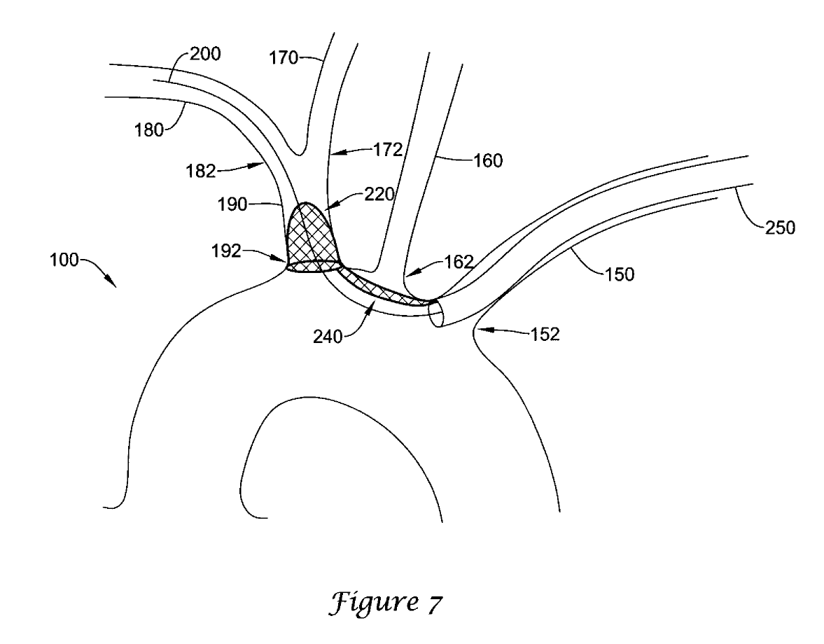

FIG. 7 illustrates, after deploying the distal filter element 220, further withdrawing the catheter 250 proximally and deploying the deflector element 240 over the ostium 162 of the left common carotid artery 160 disposed between the brachiocephalic artery 190 and the left subclavian artery 150. Due to the substantially linear arrangement of the embolic protection device 210 within the catheter 250, further proximal retraction of the catheter 250 deploys the deflector element 240 prior to deploying the proximal filter element 230.

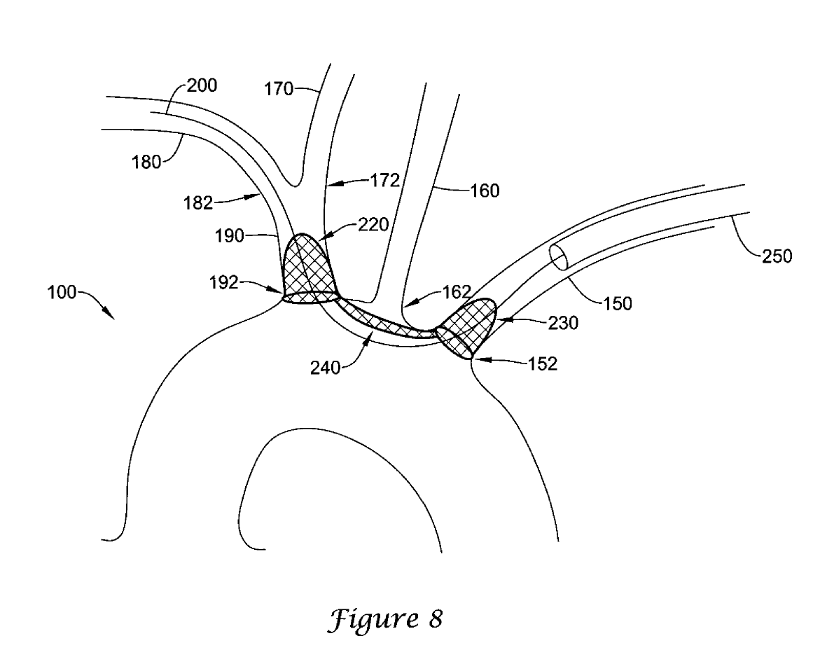

FIG. 8 illustrates a fully deployed embolic protection device 210. After deploying the deflector element 240, as shown in FIG. 7, further withdrawing the catheter 250 proximally deploys the proximal filter element 230 within the left subclavian artery 150 such that the mouth 232 of the proximal filter element 230 covers and/or overlies the ostium 152 of the left subclavian artery 150. The embolic protection device 210 may provide protection from embolic material or debris, thereby preventing embolic material from reaching the carotid arteries, while maintaining interventional or diagnostic access through the aortic arch 100. Blood flowing away from the heart and/or aortic valve at the first end 110 of the aortic arch 100 may enter and/or pass through the distal filter element 220 and the proximal filter element 230. Embolic debris carried by the blood may be trapped within the distal filter element 220 and the proximal filter element 230 by blood permeable materials 226 and 236, respectively. Similarly, blood may pass through the deflector element 240 and into the left common carotid artery 160, while embolic debris may be deflected or diverted away from the left common carotid artery 160. In some embodiments, deflected embolic debris may enter and become trapped in the proximal filter element 230, or may travel further downstream within the aorta to a location less likely to cause ischemic stroke or to another distal protection device (not shown) positioned where it will not interfere with an interventional or diagnostic procedure traversing the aortic arch 100.

In some embodiments, a method of providing embolic protection in an aortic arch 100 may further include re-advancing the catheter 250 distally over the proximal filter element 230, thereby capturing the proximal filter element 230 and embolic debris trapped therein within the catheter 250. Additionally, the method may include re-advancing the catheter 250 distally over the deflector element 240 and the distal filter element 220 thereby capturing the distal filter element 220 and embolic debris trapped therein within the catheter 250.

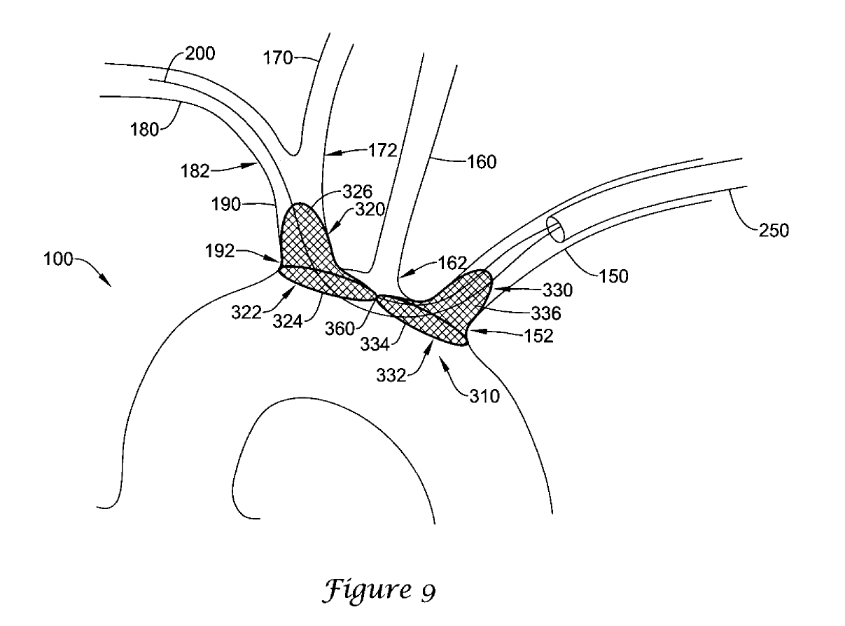

FIG. 9 illustrates an example embolic protection device 310 including a distal filter element 320 having a mouth 322 and a proximal filter element 330 having a mouth 332 deployed from a catheter 250 in the aortic arch 100 as described above with respect to embolic protection device 210. Embolic protection device 310 may be formed and/or used the same as, similar to, or in accordance with the description of embolic protection device 210 above.

In some embodiments, the distal filter element 320 is adapted to be placed in the ostium 192 of the brachiocephalic artery 190, and the mouth 322 of the distal filter element 320 is sized and adapted to cover and/or overlie the ostium 192 of the brachiocephalic artery 190 such that embolic material in the aortic arch 100 is permitted to enter the distal filter element 320 through the mouth 322 of the distal filter element 320. The distal filter element 320 may include a self-expanding support hoop 324 forming the mouth 322 and a blood permeable filter material 326 attached thereto adapted to capture the embolic material therein.

In some embodiments, the proximal filter element 330 is adapted to be placed in the ostium 152 of the left subclavian artery 150, and the mouth 332 of the proximal filter element 330 is sized and adapted to cover and/or overlie the ostium 152 of the left subclavian artery 150 such that embolic material in the aortic arch 100 is permitted to enter the proximal filter element 330 through the mouth 332 of the proximal filter element 330. The proximal filter element 330 may include a self-expanding support hoop 334 forming the mouth 332 and a blood permeable filter material 336 attached thereto adapted to capture the embolic material therein.

However, in difference to embolic protection device 210, embolic protection device 310 lacks a separate deflector element disposed between the support hoops of the distal filter element 320 and the proximal filter element 330. Instead, in embolic protection device 310, a portion of the mouth 322 of the distal filter element 320 and a portion of the mouth 332 of the proximal filter element 330 cooperate to form a deflector element, the portions being joined by a hinge element 360 disposed between the mouth 322 of the distal filter element 320 and the mouth 332 of the proximal filter element 330. The hinge element 360 may permit the mouth 322 of the distal filter element 320 and the mouth 332 of the proximal filter element 330 to open from a closed configuration, where the mouth 322 and the mouth 332 are generally mated together, to an open configuration, wherein the mouth 322 and the mouth 332 cover and/or overlie the ostium 192 and the ostium 152, respectively, the motion or translation being similar to a clamshell. Additionally, the deflector element formed by a portion of the mouth 322 of the distal filter element 320 and a portion of the mouth 332 of the proximal filter element 330 may be sized and adapted to substantially cover and/or overlie the ostium 162 of the left common carotid artery 160, and/or the ostium 172 of the right common carotid artery 170 in anatomies where the right common carotid artery 170 connects directly to the aortic arch 100.

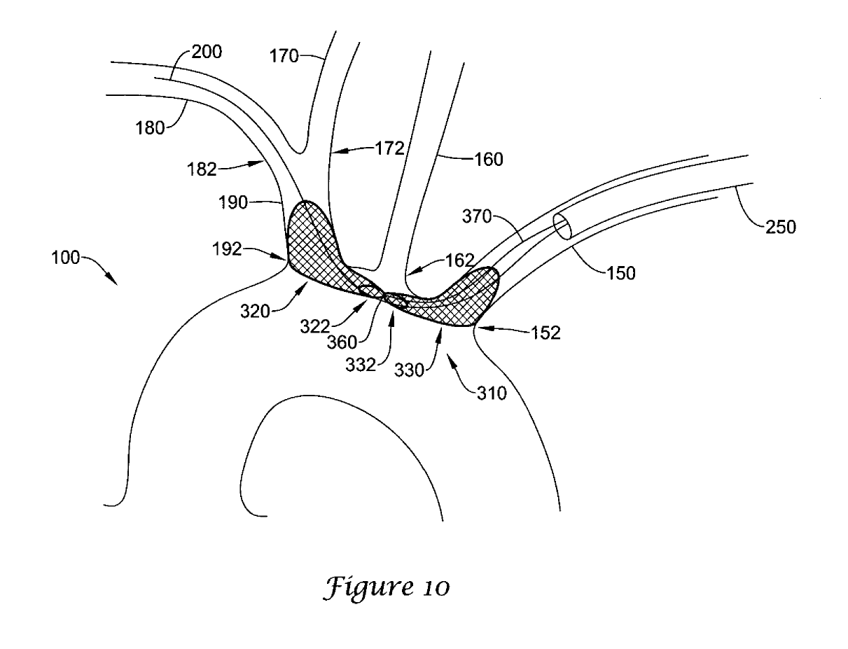

In some embodiments, the embolic protection device 310 may include a pull wire 370 configured to close the mouth 322 of the distal filter element 320 and the mouth 332 of the proximal filter element 330 at the hinge element 360 when the pull wire 370 is retracted proximally, as shown in FIG. 10. Closing the mouth 322 of the distal filter element 320 and the mouth 332 of the proximal filter element 330 captures embolic debris trapped within the distal filter element 320 and the proximal filter element 330 to facilitate re-capture and/or removal of the embolic protection device 310.

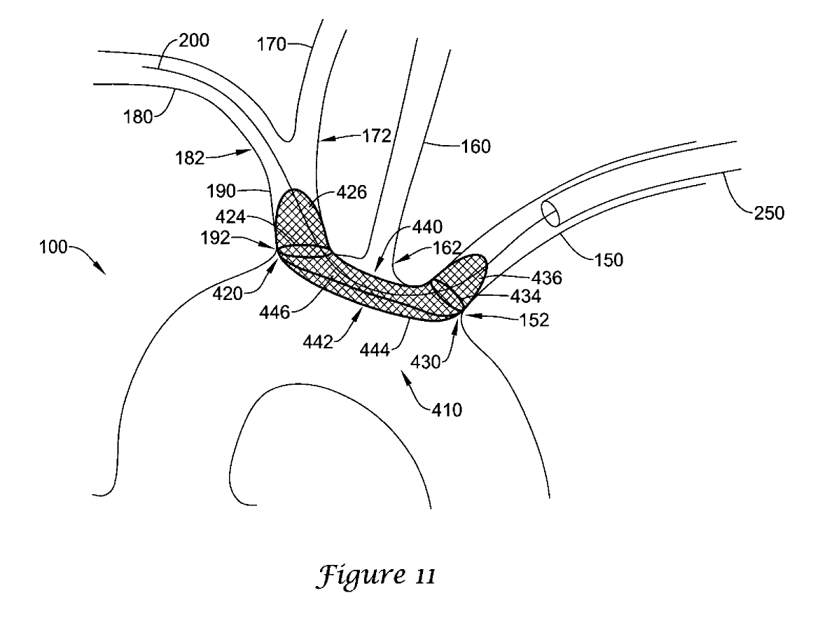

FIG. 11 illustrates an example embolic protection device 410 including a distal filter element 420, a proximal filter element 430, and a deflector element 440 disposed between and spacing apart the distal filter element 420 and the proximal filter element 430, deployed from a catheter 250 in the aortic arch 100 as described above with respect to embolic protection device 210. Embolic protection device 410 may be formed and/or used the same as, similar to, or in accordance with the description of embolic protection device 210 above. In some embodiments, the embolic protection device 410 may include a longitudinally-oriented mouth 442 extending from the distal filter element 420 to the proximal filter element 430.

In some embodiments, the distal filter element 420 is sized and adapted to be placed in the ostium 192 of the brachiocephalic artery 190. Distal filter element 420 may include a self-expanding support hoop 424 sized and adapted to cover and/or overlie the ostium 192 of the brachiocephalic artery 190 such that embolic material in the aortic arch 100 is permitted to enter the distal filter element 420. The distal filter element 420 may include a blood permeable filter material 426 attached to the self-expanding support hoop 424 and adapted to capture the embolic material therein.

In some embodiments, the proximal filter element 430 is sized and adapted to be placed in the ostium 152 of the left subclavian artery 150. Proximal filter element 430 may include a self-expanding support hoop 434 sized and adapted to cover and/or overlie the ostium 152 of the left subclavian artery 150 such that embolic material in the aortic arch 100 is permitted to enter the proximal filter element 430. The proximal filter element 430 a blood permeable filter material 436 attached to the self-expanding support hoop 434 and adapted to capture the embolic material therein.

In some embodiments, the deflector element 440 is sized and adapted to be placed over an ostium of a carotid artery disposed between the brachiocephalic artery 190 and the left subclavian artery 150, for example, the ostium 162 of the left common carotid artery 160 and/or the ostium 172 of the right common carotid artery 170 in anatomies where the right common carotid artery 170 connects directly to the aortic arch 100. The deflector element 440 may be sized to substantially cover and/or overlie the ostium 162 of the left common carotid artery 160 and/or the ostium 172 of the right common carotid artery 170.

The deflector element 440 may include a self-expanding support hoop 444 forming the longitudinally-oriented mouth 442 and a blood permeable filter material 446 attached thereto adapted to deflect or direct embolic debris away from the left common carotid artery 160. In some embodiments, deflected embolic debris may enter and become trapped in the distal filter element 420, the proximal filter element 430, or embolic debris may travel further downstream within the aorta to a location less likely to cause ischemic stroke or to another distal protection device (not shown) positioned where it will not interfere with an interventional or diagnostic procedure traversing the aortic arch 100.

In some embodiments, the longitudinally-oriented mouth 442 may be sized and configured to extend from distal the ostium 192 of the brachiocephalic artery 190 to proximal the ostium 152 of the left subclavian artery 150 such that the longitudinally-oriented mouth covers and/or overlies the ostium 192 of the brachiocephalic artery 190, the ostium 152 of the left subclavian artery 150, and the ostium 162 of the left common carotid artery 160 (and/or the ostium 172 of the right common carotid artery 170 in anatomies where the right common carotid artery 170 connects directly to the aortic arch 100) disposed between the ostium 192 of the brachiocephalic artery 190 and the ostium 152 of the left subclavian artery 150.

In some embodiments, the blood permeable filter materials 426, 436, and 446 may be formed from a single, unitary, and/or integral piece of material. In some embodiments, the self-expanding support hoop 424 of the distal filter element 420 and the self-expanding support hoop 434 of the proximal filter element 430 may be attached to, joined with, or integrally formed with the self-expanding support hoop 444. In some embodiments, the self-expanding support hoops 424, 434, and 444 may be formed as or from a single structure or piece of material.

The blood permeable filter materials of the disclosure may be formed of or include a polymeric material, a metallic or metallic alloy material, a metallic-polymer composite, combinations thereof, and the like. Examples of suitable polymers may include polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials, or other suitable materials.

The various self-expanding support hoops of the disclosure may be formed of or include a metallic material, a metallic alloy, a ceramic material, a rigid or high performance polymer, a metallic-polymer composite, combinations thereof, and the like. Some examples of some suitable materials may include metallic materials and/or alloys such as stainless steel (e.g. 304v stainless steel or 316L stainless steel), nickel-titanium alloy (e.g., nitinol, such as super elastic or linear elastic nitinol), nickel-chromium alloy, nickel-chromium-iron alloy, cobalt alloy, nickel, titanium, platinum, or alternatively, a polymer material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some embodiments, the various self-expanding support hoops of the disclosure may be mixed with, may be doped with, may be coated with, or may otherwise include a radiopaque material. Radiopaque materials are understood to be materials capable of producing a relatively bright image on a fluoroscopy screen or another imaging technique such as X-ray during a medical procedure. This relatively bright image aids the user of device in determining its location. Suitable radiopaque materials may include, but are not limited to, bismuth subcarbonate, iodine, gold, platinum, palladium, tantalum, tungsten or tungsten alloy, and the like.

In some embodiments, the medical device and/or individual components thereof may be made from, may be mixed with, may be coated with, or may otherwise include a material that provides a smooth, slick outer surface, such as but not limited to, a lubricious coating, a hydrophilic coating, a hydrophobic coating, a drug-eluting material, an anti-thrombus coating, or other suitable coating.

Although the illustrative examples described above relate to placement within the aorta for protection during heart surgery, placement in other locations is also contemplated, particularly when the preferred direction of insertion of the interventional or diagnostic device is from a point downstream of the site of the intervention and when it is desirable to avoid occlusion of the vessel lumen by filter support structures. In such an embodiment, the dimensions of the filter element(s) and the lengths of the filter wire and delivery catheter may be adjusted to better suit the deployment site.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

We claim:

1. A medical device for aortic embolic protection, comprising:
a guidewire;
an embolic protection device slideably disposed about the guidewire, the embolic protection device including a distal filter element having a mouth, a proximal filter element having a mouth, and a deflector element disposed between and spacing apart the mouth of the distal filter element and the mouth of the proximal filter element; and
a catheter configured to receive the embolic protection device therein, the catheter being slideably disposed over the guidewire.

2. The medical device of claim 1, wherein the distal filter element is adapted to be placed in an ostium of a brachiocephalic artery, the proximal filter element is adapted to be placed in an ostium of a left subclavian artery, and the deflector element is adapted to be placed over an ostium of a carotid artery disposed between the brachiocephalic artery and the left subclavian artery.

3. The medical device of claim 2, wherein proximal retraction of the catheter deploys the distal filter element prior to deploying the deflector element.

4. The medical device of claim 3, wherein further proximal retraction of the catheter deploys the deflector element prior to deploying the proximal filter element.

5. The medical device of claim 2, wherein the mouth of the distal filter element is adapted to cover the ostium of the brachiocephalic artery, such that embolic material is permitted to enter the distal filter element through the mouth of the distal filter element.

6. The medical device of claim 2, wherein the mouth of the proximal filter element is adapted to cover the ostium of the left subclavian artery, such that embolic material is permitted to enter the proximal filter element through the mouth of the proximal filter element.

7. The medical device of claim 2, wherein the deflector element is adapted to cover the ostium of the carotid artery, thereby preventing embolic material from entering the carotid artery.

8. The medical device of claim 1, wherein the catheter is configured to receive the embolic protection device therein in a substantially linear arrangement.

9. The medical device of claim 1, wherein the mouth of the distal filter element includes a self-expanding support hoop.

10. The medical device of claim 1, wherein the mouth of the proximal filter element includes a self-expanding support hoop.

11. The medical device of claim 1, wherein a portion of the mouth of the distal filter element and a portion of the mouth of the proximal filter element cooperate to form the deflector element, the portions being joined by a hinge element disposed between the mouth of the distal filter element and the mouth of the proximal filter element.

12. The medical device of claim 11, wherein the embolic protection device includes a pull wire configured to close the mouth of the distal filter element and the mouth of the proximal filter element when the pull wire is retracted proximally.

13. A medical device for aortic embolic protection, comprising:
a guidewire;
an embolic protection device slideably disposed about the guidewire, the embolic protection device including a distal filter element, a proximal filter element, and a deflector element disposed between and spacing apart the distal filter element and the proximal filter element;
wherein the embolic protection device includes a longitudinally-oriented mouth extending from the distal filter element to the proximal filter element; and
a catheter configured to receive the embolic protection device therein, the catheter being slideably disposed over the guidewire.

14. The medical device of claim 13, wherein the longitudinally-oriented mouth includes a self-expanding support hoop.

15. The medical device of claim 13, wherein the longitudinally-oriented mouth is sized and configured to extend from distal an ostium of a brachiocephalic artery to proximal an ostium of a left subclavian artery such that the longitudinally-oriented mouth covers the ostium of the brachiocephalic artery, the ostium of the left subclavian artery, and an ostium of a carotid artery disposed between the ostium of the brachiocephalic artery and the ostium of the left subclavian artery.

16. A method of providing embolic protection in an aortic arch, comprising:
inserting a guidewire from a left subclavian artery through the aortic arch into a brachiocephalic artery;
advancing a catheter having an embolic protection device disposed therein over the guidewire from the left subclavian artery through the aortic arch into the brachiocephalic artery, the embolic protection device including a distal filter element having a mouth, a proximal filter element having a mouth, and a deflector element disposed between and spacing apart the mouth of the distal filter element and the mouth of the proximal filter element;
withdrawing the catheter proximally and deploying the distal filter element within the brachiocephalic artery such that the mouth of the distal filter element overlies an ostium of the brachiocephalic artery;
after deploying the distal filter element, further withdrawing the catheter proximally and deploying the deflector element over an ostium of a carotid artery disposed between the brachiocephalic artery and the left subclavian artery; and
after deploying the deflector element, further withdrawing the catheter proximally and deploying the proximal filter element within the left subclavian artery such that the mouth of the proximal filter element overlies an ostium of the left subclavian artery.

17. The method of claim 16, further comprising re-advancing the catheter distally over the proximal filter element thereby capturing the proximal filter element and embolic debris trapped therein within the catheter.

18. The method of claim 17, further comprising re-advancing the catheter distally over the deflector element and the distal filter element thereby capturing the distal filter element and embolic debris trapped therein within the catheter.
